(19) United States
(12) Patent Application Publication
(10) Pub. No.: US 2026/0192095 A1
(43) Pub. Date: Jul. 9, 2026

(54) DEVICE FOR HOLDING POST-SURGERY DRAIN RESERVOIR

(71) Applicants: Elizabeth Benditt, Overland Park, KS (US); Howard C. Weiner, Overland Park, KS (US)

(72) Inventors: Elizabeth Benditt, Overland Park, KS (US); Howard C. Weiner, Overland Park, KS (US)

(21) Appl. No.: 19/551,114

(22) Filed: Feb. 26, 2026

Related U.S. Application Data
(60) Provisional application No. 63/785,943, filed on Apr. 9, 2025.

(51) Int. Cl. A61M 27/00

(52) U.S. Cl. CPC A61M 27/00; A61M 2209/088

(57) ABSTRACT

A device is provided for holding a post-operative bodily fluid drain reservoir with an attachment loop. The device includes a cord and a reservoir coupling device. The cord defines a first loop for extending around a neck of a patient. The reservoir coupling device is operatively associated with the cord and is configured to releasably couple to the attachment loop of the post-operative bodily fluid drain reservoir.

### CROSS-REFERENCE TO RELATED APPLICATIONS

The current patent application is a non-provisional utility patent application which claims priority benefit of earlier-filed U.S. Provisional Application Ser. No. 63/785,943; titled “POST-SURGERY DRAIN RESERVOIR HOLDER”; and filed Apr. 9, 2025. The Provisional Application is hereby incorporated by reference, in its entirety, into the current patent application.

### BACKGROUND OF THE INVENTION

Post-operative drain reservoirs, such as for Jackson-Pratt (JP) drain systems, are often used in surgeries where there is significant potential for fluid accumulation, which could lead to complications such as infection or impaired healing. Such surgeries may include mastectomies, hernia repairs, pleural effusion, thyroidectomy, plastic surgery, lymphadenectomy, or the like. Such a drain system may include a drainage tube with a drainage end positioned in the patient's body, a length of flexible tubing connected to the drainage end and extending out of the patient's body at an insertion site defined by an incision in the patient's skin, and a reservoir connected to the tubing and that collects bodily fluids, such as lymphatic fluid and blood. The reservoir is generally a flexible bulb that is squeezed to apply a negative pressure to pull the fluids from the wound, through the tubing, and into the reservoir.

The reservoir is generally made of a plastic material sufficiently thick and resilient to form the needed negative pressure, which adds to its weight. Additionally, as the reservoir fills with bodily fluids, the weight of the reservoir increases. Even though the incision may be supported by one or more sutures and or bandages, the weight of the reservoir on the incision is painful. Thus, the reservoir often needs to be supported to remove the weight from the incision. A current solution is to use a safety pin to connect the reservoir to clothing. However, it can feel uncomfortable or embarrassing for a patient to wear their reservoir on their outer clothing. Additionally, the reservoir cannot be removed along with the clothes when the patient must shower, bathe, or change clothes, for example. Another current solution is to use a band that wraps laterally around the patient's torso to hold the reservoir. However, the band is often difficult to put on and remove after surgery when the patient is sore and uncomfortable or restrictive to wear. Additionally, the band is similarly insufficient in the context of showers or baths.

Thus, there is a need for an improved means for supporting the reservoir of a drainage system. This background discussion is intended to provide information related to the present invention which is not necessarily prior art.

### SUMMARY OF THE INVENTION

Embodiments of the current invention address one or more of the above-mentioned problems and provide a distinct advance in the art of means for supporting post-operative bodily fluid drain reservoirs.

One embodiment of the invention is a device for holding a post-operative bodily fluid drain reservoir with an attachment loop. The device includes a cord and a reservoir coupling device. The cord defines a first loop for extending around a neck of a patient. The reservoir coupling device is operatively associated with the cord and is configured to releasably couple to the attachment loop of the post-operative bodily fluid drain reservoir.

A post-operative bodily fluid drain reservoir device according to another embodiment of the invention includes a cord, a coupling device, and a post-operative bodily fluid drain reservoir. The cord defines a first loop for extending around a neck of a patient and includes a buckle for adjusting a length of the first loop. The coupling device is operatively associated with the cord and presents a first end, a body portion extending from the first end to define a coupling loop, and a second end connected to the body portion. The first end is shiftable between a first position in which the first end is proximal to the second end and a second position in which the first end is distal relative to the second end to define a gap. The post-operative bodily fluid drain reservoir has an attachment loop operable to be inserted through the gap of the coupling device to secure the post-operative bodily fluid drain reservoir to the cord.

A device for holding a post-operative bodily fluid drain reservoir with an attachment loop according to another embodiment of the invention includes a cord, a coupling device, a magnet, and magnetic material. The cord defines a loop for extending around a neck of a patient. The coupling device is operatively associated with the cord and presents a first end, a body portion extending from the first end to define a coupling loop, and a second end connected to the body portion proximal to the first end. A portion of the coupling device is formed of flexible material so that the first and second ends are operable to be shifted away from one another to define a gap for receiving a portion of the attachment loop. The magnet is embedded in the first end, and the magnetic material is embedded in the second end. The magnet and magnetic material help hold the first and second ends of the coupling device proximal to one another to help support the weight of the post-operative bodily fluid drain reservoir.

A device for holding a post-operative bodily fluid drain reservoir with an attachment loop according to another embodiment of the invention includes a cord, a coupling device, a magnet, and magnetic material. The cord defines a loop for extending around a neck of a patient. The coupling device is operatively associated with the cord and presents a first end, a body portion extending from the first end to define a coupling loop, and a second end connected to the body portion. The first end is shiftable between a first position in which the first end is proximal to the second end and a second position in which the first end is distal relative to the second end to define a gap for receiving a portion of the attachment loop. The magnet is attached to the first end, and the magnetic material is attached to the second end so that a magnetic field of the magnet biases the first end to the first position.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the current invention will be apparent from the following detailed description of the embodiments and the accompanying drawing figures.

### BRIEF DESCRIPTION OF DRAWINGS

Embodiments of the current invention are described in detail below with reference to the attached drawing figures, wherein:

FIG. 1 is perspective view of a device for supporting a post-operative bodily fluid drain reservoir according to an embodiment of the invention;

FIG. 2 is a perspective view of an exemplary coupling device of the device of FIG. 1;

FIG. 3 is a sectional view of the coupling device of FIG. 2 as viewed along lines 3-3;

FIG. 4 is a view of the coupling device of FIG. 2 with first and second ends positioned at a distance to one another;

FIG. 5 is a view of the coupling device of FIG. 4 with an attachment loop of the post-operative bodily fluid drain reservoir being looped onto the coupling device; and

FIG. 6 is a view of the coupling device of FIG. 2 with the first and second ends position proximal to one another, thereby securing the post-operative bodily fluid drain reservoir to the coupling device.

The drawing figures do not limit the current invention to the specific embodiments disclosed and described herein. The drawings are not necessarily to scale, emphasis instead being placed upon clearly illustrating the principles of the invention.

### DETAILED DESCRIPTION OF THE INVENTION

The following detailed description of the technology references the accompanying drawings that illustrate specific embodiments in which the technology can be practiced. The embodiments are intended to describe aspects of the technology in sufficient detail to enable those skilled in the art to practice the technology. Other embodiments can be utilized and changes can be made without departing from the scope of the current invention. The following detailed description is, therefore, not to be taken in a limiting sense. The scope of the current invention is defined only by the appended claims, along with the full scope of equivalents to which such claims are entitled.

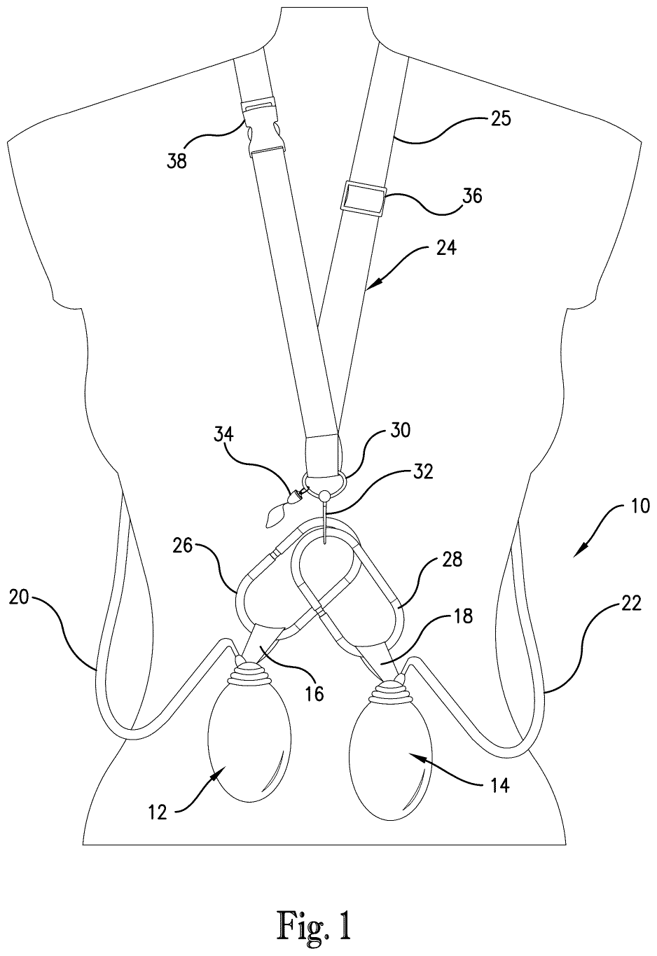

Turning to FIG. 1, a device 10 for holding one or more post-operative bodily fluid drain reservoirs 12, 14 is depicted. The device 10 is configured to support the post-operative bodily fluid drain reservoirs 12, 14 in a comfortable manner that may be hidden from view by other people underneath one or more layers of clothes. The post-operative bodily fluid drain reservoirs 12, 14 may include any drain reservoirs known in the art, including JP drain system reservoirs. The reservoirs 12, 14 may include attachment loops 16, 18 and drain tubes 20, 22 in fluid communication with the reservoirs 12, 14. In one or more embodiments, the device 10 broadly comprises one or more cord 24 and one or more coupling devices 26, 28.

The cord 24 defines a first loop 25 for extending around the neck of a patient. The loop may have any shape without departing from the scope of the present invention. For example, the loop may be continuous or discontinuous, closed or open, curved, rectilinear, polygonal shaped, and/or the like. The cord 24 may be made of a highly flexible material, such as string, clothe, or the like, or combinations thereof. In one or more embodiments, the cord 24 is a lanyard. The cord 24 may be sized so that the coupling devices 26, 28 can hang below the cord 24 and temporarily engage post-operative drain reservoirs 12, 14. While the cord 24 is depicted as being a substantially thin and flat shape, the cord 24 may have any shape without departing from the scope of the present invention.

In one or more embodiments, cord 24 includes one or more intermediate coupling device 30, 32 configured to connect the reservoir coupling devices 26, 28 to the cord 24. The intermediary coupling devices 30, 32 may include a D-shaped ring 30 or keychain ring and a swiveling clip 32 rotatably connected to the ring 30. Additionally or alternatively, the reservoir coupling devices 26, 28 may be connected directly to the cord 24. The shape of the cord 24 and swiveling clip 32 enable the coupling devices 26, 28 to rest substantially flat against the patient's body and the reservoirs 12, 14 to freely rotate and move, thereby reducing tugs against the tender areas where drains are sewn into the body.

In one or more embodiments, the cord 24 defines a second loop connected to the first loop, such as a lanyard breakaway connector 34. The cord 24 may further include one or more length-adjustment devices 36, such as a buckle or the like, for adjusting the circumference of the loop 25 and therefore the location of the coupling devices 26, 28 to raise or lower the reservoirs 12, 14 at different positions relative to the patient's body. The cord 24 may also include one or more disconnect devices 38 configured to open the first loop 25, such as a quick-release buckle, slide-release buckle, or the like.

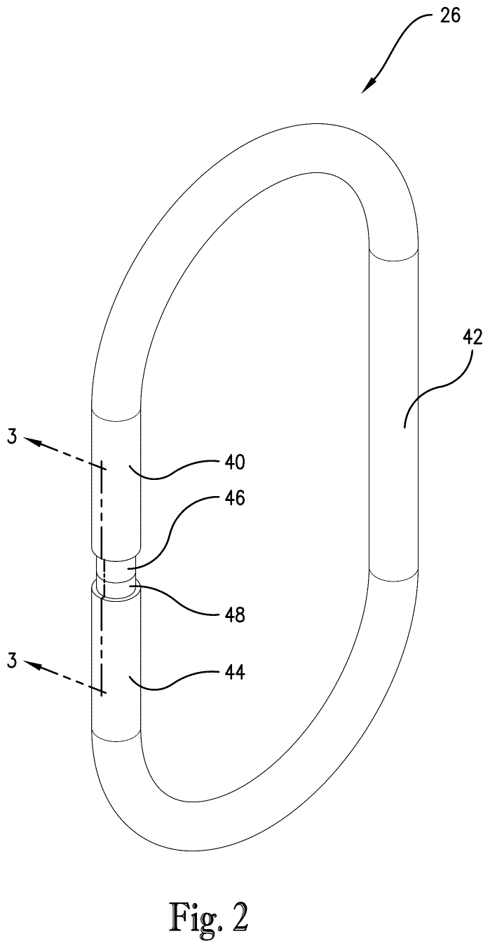

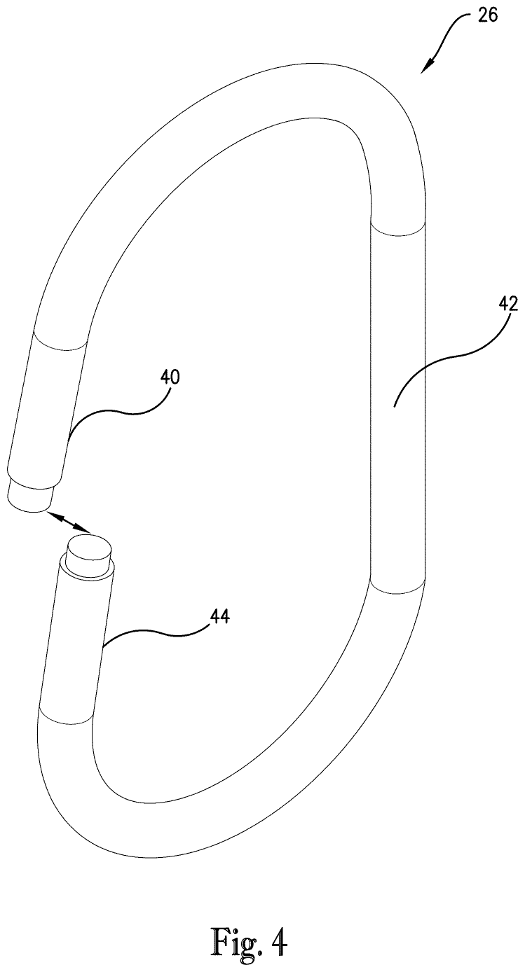

Turning to FIG. 2, a representative coupling device 26 is depicted. The other coupling device 28 may include substantially similar components. The coupling device 26 can be operatively associated with the cord 24 and is configured to releasably couple to one or more attachment loops 16, 18 of the one or more post-operative bodily fluid drain reservoirs 12, 14. The coupling device 26 presents a first end 40, a body portion 42 extending from the first end 40 to define a coupling loop, and a second end 44 connected to the body portion 42 proximal to the first end 40. The loop may have any shape without departing from the scope of the present invention. For example, the loop may be continuous or discontinuous, closed or open, curved, rectilinear, polygonal shaped, and/or the like. One or more of the ends 40, 44 are shiftable between a first relative orientation (depicted in FIG. 2) in which the ends 40, 44 are proximal to one another and a second relative orientation (depicted in FIG. 4) in which the ends 40, 44 are distal relative to one another, thereby defining a gap for receiving a portion of the attachment loop of a reservoir. The coupling device 26 may define a generally loop-shaped structure, such as a c-shape, so that one or more attachment loops of the reservoirs can be hooked or looped around the coupling device 26 through the gap formed between the ends 40, 44. While the figures depict the reservoir coupling device 26 comprising c-shaped clips, the reservoir coupling device 26 may be any device configured to temporarily couple the reservoirs to the cord without departing from the scope of the present invention. Additionally, the coupling device 26 may have any number of shapes without departing from the scope of the present invention.

In one or more embodiments, the coupling device 26 includes one or more magnetic materials 46, 48 operatively associated with the first and second ends 40, 44 for helping maintain the first and second ends 40, 44 in the first relative orientation. In one or more embodiments, at least one of the magnetic materials 46 comprises a permanent magnet, and the other magnetic material 48 comprises a material with magnetic susceptibility, such as iron, stainless steel, or the like, or combinations thereof. In one or more embodiments, both magnetic materials 46, 48 comprise permanent magnets.

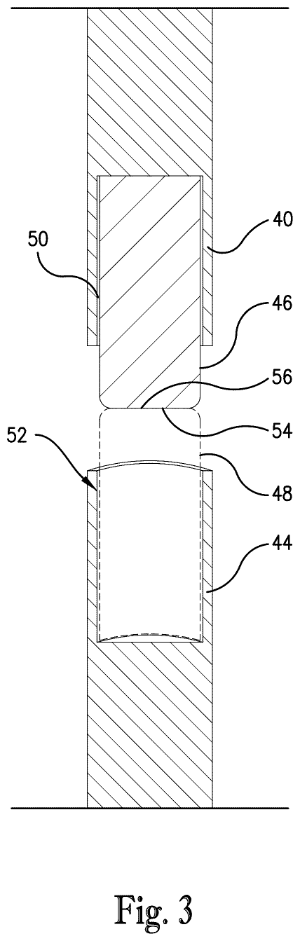

Turning to FIG. 3, in one or more embodiments, the first magnetic material 46 is attached to the first end 40, and the second magnetic material is attached to the second end 44. The magnetic materials 46, 48 may be at least partially inset or embedded within their respective ends 40, 44. In one or more embodiments, the magnetic materials 46, 48 are secured to their respective ends 40, 44 via adhesive or other suitable attachment means. The magnetic materials 46, 48 are selected and arranged so that they are attracted to one another to bias the first and second ends 40, 44 in the first relative orientation, or proximal to one another. For example, the magnetic material 48 attached to the second end 44 is selected so that a magnetic field of the magnet 46 of the first end 40 biases the ends 40, 44 to the first relative orientation. In one or more embodiments where the magnetic materials 46, 48 are both magnets, the magnets 46, 48 are oriented so that opposite magnetic polarities of the magnets 46, 48 face one another so that the first and second magnets 46, 48 are attracted to one another, thereby helping the two ends 40, 44 remain proximal to one another.

In one or more embodiments, the first end 40 includes a first cavity 50 that houses the first magnetic material 46, and the second end 44 includes a second cavity 52 that houses the second magnetic material 48. The cavities 50, 52 extend axially into their respective ends 40, 44. In one or more embodiments, portions of the magnetic materials 46, 48 protrude from their respective ends 40, 44 toward one another when the ends 40, 44 are in the first proximal relative orientation. The magnetic materials 46, 48 may be cylindrically shaped with circular surfaces 54, 56 facing one another when the ends 40, 44 are in the first proximal relative orientation. In one or more embodiments, the magnetic materials 46, 48 contact one another when the two ends 40, 44 are in the first relative orientation. For example, the surfaces 54, 56 of the magnetic materials 46, 48 contact one another when the ends 40, 44 are in the first relative orientation. In one or more embodiments, the magnetic materials 46, 48 have diameters of about 4 millimeters (mm) and lengths of about 4 mm. The magnetic materials 46, 48 may be embedded about 3 mm into their respective ends 40, 44, thereby leaving about 1 mm exposed. However, the magnetic materials 46, 48 may have any number of sizes or dimensions and be embedded into their respective ends 40, 44 at any number of depths without departing from the scope of the present invention. In one or more embodiments, the magnetic materials 46, 48 are simply adhered to their respective ends 40, 44 and are not embedded into their respective ends 40, 44. The magnetic materials 46, 48 enable the patient to easily open the coupling device 26 to readily remove any reservoirs, such as for emptying and/or measuring the fluid therein. The magnetic materials 46, 48 also enable easy closure of the coupling device 26 such that the coupling device 26 can be opened and closed with one hand while providing security that the reservoirs will stay in place.

The magnetic materials 46, 48 are selected and arranged so that the magnetic forces thereof help bias the coupling device 26 to the closed position (or first relative proximal orientation of the ends 40, 44) and remain closed, but are easily pulled apart easily with one hand. This provides the patient with additional confidence that the reservoirs will stay in place and provide a simple and intuitive means to open the coupling device 26 up to remove and empty reservoirs as needed.

Turning to FIG. 4, in one or more embodiments, at least a portion of the coupling device 26 is formed of flexible material so that the first and second ends 40, 44 are operable to be shifted away from one another to the second relative orientation to define the gap for receiving a portion of the attachment loop of the reservoir. For example, the first end 40, the body portion 42, and/or the second end 44 may be made of the flexible material. In one or more embodiments, at least a portion of the coupling device 26 is formed of resilient material so that the first and second ends 40, 44 are biased by the resilience of the material to the first proximal relative orientation. For example, the first end 40, the body portion 42, and/or the second end 44 may be made of the resilient material. The flexible and resilient material may comprise polymers, such as plastic, elastomers, rubber, fibers, metal, or the like, or combinations thereof. The coupling device 26 may have a generally smooth surface to reduce scratching or hurting the patient's body with a sharp edge. While the device 10 is depicted as having the coupling devices 26, 28 described herein, the device 10 may additionally or alternatively include any number of coupling devices, including carabiners, plastic snaps, or the like, without departing from the scope of the present invention.

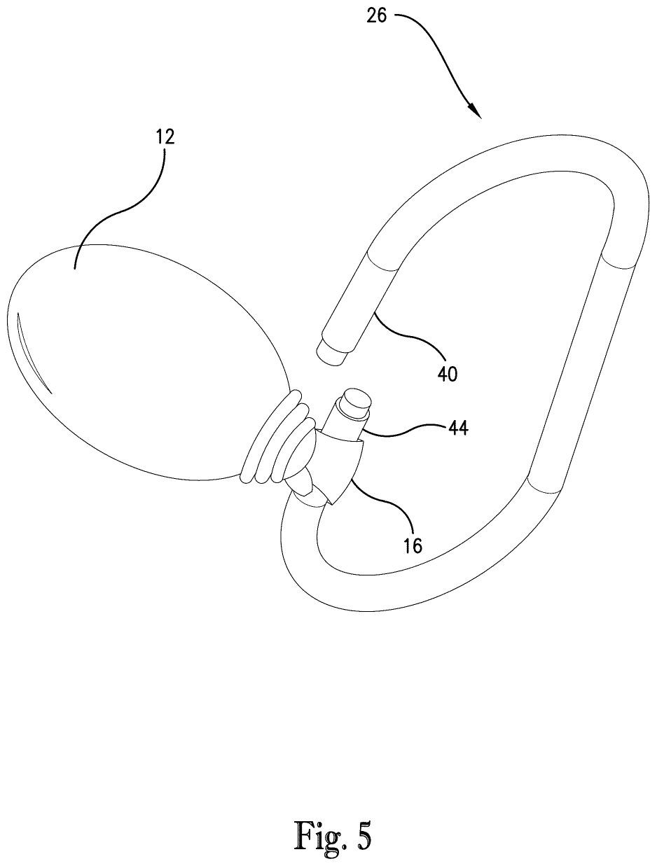

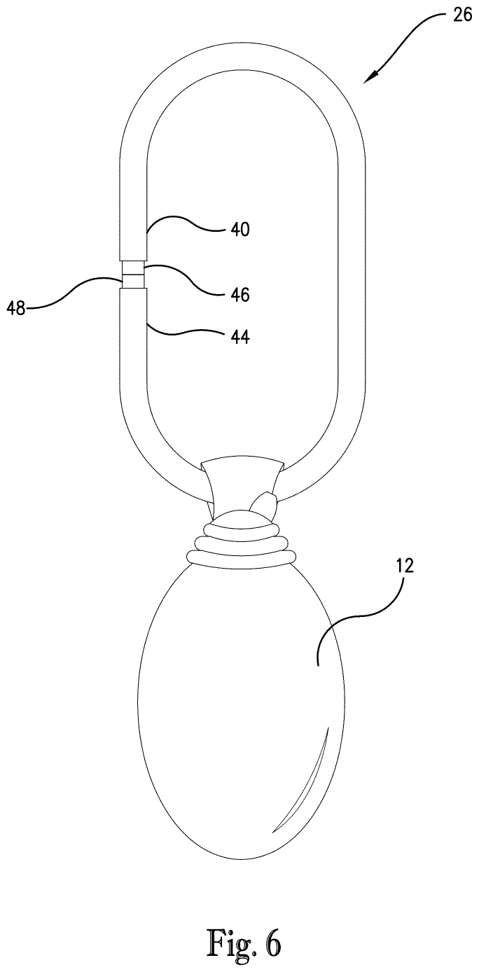

Turning to FIG. 5, in use, one or more of the ends 40, 44 are manipulated to overcome the magnetic forces between the magnetic materials 46, 48 to shift the ends 40, 44 from the first relative orientation to the second relative orientation so that a portion of the attachment loop 16 is inserted through the gap between the ends 40, 44 of the coupling device 26. One of the ends 44 is inserted through the attachment loop 16 of one of the drain reservoirs 12 while the ends 40, 44 are held in the second relative orientation. Similarly, one of the ends 40, 44 of the coupling device 26 can be inserted through the loop of one of the intermediate coupling devices to secure the coupling device 26 to the cord. As depicted in FIG. 6, when the patient releases the ends 40, 44 of the coupling device 26, the resilience of the materials of the coupling device 26 and the magnetic materials 46, 48 cause the ends 40, 44 of the coupling device 26 to return to the first relative orientation so that the reservoir 12 is secured to the coupling device 26 and the cord.

Throughout this specification, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the current invention can include a variety of combinations and/or integrations of the embodiments described herein.

Although the present application sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than or equal to about 10” (with no upper bounds) and a claim reciting “less than or equal to about 100” (with no lower bounds).

Furthermore, unless otherwise specified, any directional references (e.g., upper, lower, above, below, etc.) are used herein solely for the sake of convenience and should be understood only in relation to each other. For instance, a component might in practice be oriented such that faces referred to as “upper” and “lower” are sideways, angled, inverted, etc. relative to the chosen frame of reference.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The patent claims at the end of this patent application are not intended to be construed under 35 U.S.C. § 112(f) unless traditional means-plus-function language is expressly recited, such as “means for” or “step for” language being explicitly recited in the claim(s).

Although the technology has been described with reference to the embodiments illustrated in the attached drawing figures, it is noted that equivalents may be employed and substitutions made herein without departing from the scope of the technology as recited in the claims.

Having thus described various embodiments of the technology, what is claimed as new and desired to be protected by Letters Patent includes the following:

1. A post-operative bodily fluid drain reservoir device comprising:
one or more cord defining a first loop for extending around a neck of a patient and including one or more buckle for adjusting a length of the first loop;
one or more coupling device operatively associated with the one or more cord and presenting a first end, a body portion extending from the first end to define a coupling loop, and a second end connected to the body portion, the first end being shiftable between a first position in which the first end is proximal to the second end and a second position in which the first end is distal relative to the second end to define a gap; and
a post-operative bodily fluid drain reservoir having one or more attachment loop operable to be inserted through the gap of the one or more coupling device to secure the post-operative bodily fluid drain reservoir to the one or more cord.

2. The post-operative bodily fluid drain reservoir device of claim 1, wherein the first loop is a closed loop.

3. The post-operative bodily fluid drain reservoir device of claim 1, wherein the one or more coupling device is substantially c-shaped.

4. The post-operative bodily fluid drain reservoir device of claim 1, wherein the one or more coupling device is made of a resilient material configured to return to its original shape after being bent.

5. The post-operative bodily fluid drain reservoir device of claim 1, further comprising a first magnet embedded in a first end of the two ends and a second magnet embedded in a second end of the two ends, the first and second magnets being oriented so that opposite magnetic polarities face one another so that the first and second magnets are attracted to one another, thereby helping the two ends of the one or more coupling device remain proximal to one another.

6. The post-operative bodily fluid drain reservoir device of claim 5, wherein the first end includes a first cavity that houses the first magnet, and the second end includes a second cavity that houses the second magnet.

7. The post-operative bodily fluid drain reservoir device of claim 6, wherein the first cavity extends axially into the first end, and the second cavity extends axially into the second end.

8. The post-operative bodily fluid drain reservoir device of claim 5, wherein a portion of the first magnet protrudes from the first end toward the second end, and a portion of the second magnet protrudes from the second end toward the first end.

9. The post-operative bodily fluid drain reservoir device of claim 5, wherein the first and second magnets are cylindrical permanent magnets.

10. The post-operative bodily fluid drain reservoir device of claim 5, wherein the first magnet contacts the second magnet when the two ends are proximal to one another.

11. A device for holding a post-operative bodily fluid drain reservoir with an attachment loop, the device comprising:
a cord defining a loop for extending around a neck of a patient;
a coupling device operatively associated with the cord and presenting a first end, a body portion extending from the first end to define a coupling loop, and a second end connected to the body portion proximal to the first end, at least a portion of the coupling device formed of flexible material so that the first and second ends are operable to be shifted away from one another to define a gap for receiving a portion of the attachment loop;
a magnet embedded in the first end; and
a magnetic material embedded in the second end.

12. The device of claim 11, wherein the magnet is a first permanent magnet, and the magnetic material is a second permanent magnet that is oriented so that a magnetic field of the second permanent magnet attracts the first permanent magnet, thereby helping retain the first end proximal to the second end.

13. The device of claim 11, wherein the portion of the coupling device is formed of a resilient material to bias the coupling device to bias the first end in proximal relation to the second end.

14. The device of claim 11, wherein the first end includes a first cavity that houses the magnet, and the second end includes a second cavity that houses the magnetic material.

15. The device of claim 14, wherein at least a portion of the magnet protrudes from the first cavity towards the second end when the first and second ends are proximal to one another.

16. The device of claim 11, wherein the coupling device is a first coupling device and the post-operative bodily fluid drain reservoir is a first post-operative bodily fluid drain reservoir, further comprising a second coupling device operatively associated with the cord and presenting a first end, a body portion extending from the first end to define a coupling loop, and a second end connected to the body portion proximal to the first end, at least a portion of the coupling device formed of flexible, resilient material so that the first and second end are operable to be shifted away from one another to define a gap for receiving a portion of an attachment loop of a second post-operative bodily fluid drain reservoir and shift back to a shape in which the first and second ends of the second coupling device are proximal to one another.

17. A device for holding a post-operative bodily fluid drain reservoir with an attachment loop, the device comprising:
a cord defining a loop for extending around a neck of a patient;
a coupling device operatively associated with the cord and presenting a first end, a body portion extending from the first end to define a coupling loop, and a second end connected to the body portion, the first end being shiftable between a first position in which the first end is proximal to the second end and a second position in which the first end is distal relative to the second end to define a gap for receiving a portion of the attachment loop;
a magnet attached to the first end; and
a magnetic material attached to the second end so that a magnetic field of the magnet biases the first end to the first position.

18. The device of claim 17, wherein the magnet is a cylindrical magnet with a circular surface facing the second end when the first end is in the first position.

19. The device of claim 18, wherein the magnet is at least partially inset in the first end.

20. The device of claim 19, wherein the circular surface of the magnet contacts the magnetic material when the first end is in the first position.
